(19) United States
(12) Patent Application Publication
(10) Pub. No.: US 2016/0120775 A1
(43) Pub. Date: May 5, 2016

(54) METHOD FOR PRODUCING A TATTOO INK

(71) Applicant: Andreas Wampl, Dornbirn (AT)

(72) Inventor: Andreas Wampl, Dornbirn (AT)

(21) Appl. No.: 14/895,312

(22) PCT Filed: May 16, 2014
(86) PCT No.: PCT/AT2014/000111

§ 371 Date: Dec. 2, 2015

(30) Foreign Application Priority Data
Jun. 6, 2013 (AT) A 460/2013

(51) Int. Cl. A61K 8/19; A61Q 1/02

(52) U.S. Cl. CPC A61K 8/19; A61Q 1/02

(57) ABSTRACT

A method for producing a tattoo ink, in which a carbon extracted from vegetable and/or animal and/or human material is added to a base composition of the tattoo ink.

### BACKGROUND

The invention relates to a method for producing a tattoo ink, and also a tattoo ink itself and also a tattoo method.

Tattoos are currently very common and are principally used for body decoration. By having tattoos of names, pictures and the like, tattoos, however, frequently also are intended to express a particularly close relationship to another person, an animal or the like.

### SUMMARY

It is the object of the invention to provide a technical possibility with which a tattoo in a particular manner can produce or express a close relationship to another person, an animal or else a plant.

In a method for producing a tattoo ink, for this purpose according to the invention it is provided that carbon obtained from plant and/or animal and/or human material is added to a base composition of the tattoo ink. For the addition, known or usual stirring or other mixing methods can be used. The tattoo ink thus produced therefore has a specific carbon fraction which is obtained from plant and/or animal and/or human material, wherein the origin of the carbon may be clearly assigned to a defined person, a defined animal and/or a defined plant. The tattoo produced using this tattoo ink therefore has the special feature that it contains carbon that originates from an individual plant, a defined animal, or especially, also from a defined human. It is possible hereby to express a particularly close attachment to, and also memory of, a certain plant, a certain animal, or a certain human. By corresponding documentation, a clear proof of origin of the carbon in the tattoo ink can be supplied. The method according to the invention, comprises, in a base variant, first only the mixing of the base composition with the correspondingly obtained carbon. Clearly, carbon can also be obtained from various defined sources and added to the base composition of the tattoo ink. In this case, starting materials for obtaining the carbon that do not permit individual assignment of the carbon to the source thereof are not co-comprised in the preferred embodiments of the invention.

### DETAILED DESCRIPTION OF THE PREFERRED EMBODIMENT

In particularly preferred embodiments of the invention, the method according to the invention comprises, in addition to said addition, also the obtaining of the carbon. For this purpose, these preferred variants of the method provide that the carbon is obtained from plant and/or animal and/or human material before it is added to the base composition. For obtaining the carbon, use can be made of chemical extraction methods that are known per se. The material can also be carbonized in order to obtain the carbon.

Expediently, the carbon obtained each time is mechanically pulverized. It is therefore expediently provided that the carbon obtained from plant and/or animal and/or human material is added to the base composition as powder or as a powder-liquid mixture.

Before the carbon is obtained, the corresponding material is taken in preferred embodiments from the plant and/or the animal and/or the human. The starting material from which the carbon is produced is preferably dead material. Examples of such material as carbon source are, in the case of humans, hair, nails, skin, cornea, tooth material and/or the like. In the case of animals, as corresponding starting material, e.g. hair, nails, claw material, hoof material, hide, leather, tooth material and/or horn can be used. Preferably, it is therefore provided that the animal and/or human, preferably dead, material comprises hair and/or nails and/or claw material and/or hoof material and/or skin and/or leather and/or tooth material and/or horn or consists thereof. In the case of plants, use can be made of corresponding plant components such as leaves, branches, bark, roots and the like, in order to obtain carbon therefrom. Of course, there are still more possibilities of corresponding starting materials for obtaining carbon.

The base composition can be a liquid, a liquid mixture, an emulsion, that is to say a mixture of non-mutually-soluble liquids, or a mixture of at least one liquid or emulsion and at least one solid. For example, familiar commercially available tattoo pigments or other tattoo inks, in particular also in various colors, can be used as base composition, in order to add thereto, according to the invention, the individual carbon obtained from the respective plant and/or the respective animal and/or the respective human.

Carbon is preferably elemental carbon having the chemical symbol C, which, as is known, can occur in various stable and unstable isotopes. Particularly preferably, the carbon obtained from the plant and/or animal and/or human material is present at least in part as graphite or as charcoal, or as amorphous carbon, in order then to be added in preferred embodiments in powder form or powder-liquid mixture to the base composition. The carbon obtained is expediently highly pure carbon. In practice, here, generally a degree of purity of 100% cannot be achieved. Therefore, expressed more precisely it is a mixed material which contains the carbon that is obtained. In the sense of a purity as high as possible, however, it is expediently provided that the carbon that is obtained from plant and/or animal and/or human material makes up at least 70% by weight, preferably at least 99% by weight, of a mixed material which is added to the base composition. The carbon thus obtained can have a relatively high weight fraction of the completed tattoo ink. Generally, it is expediently provided that the carbon obtained from plant and/or animal and/or human material is added to the base composition at a fraction of 0.1% by weight to 50% by weight of the tattoo ink.

In addition to the method for producing the tattoo ink, the invention also relates to the tattoo ink per se. It is in this case a tattoo ink that is characterized in that carbon obtained from plant and/or animal and/or human material is added to a base composition of the tattoo ink. Expediently, the tattoo ink according to the invention is produced by a method according to the invention.

In the case of tattoo methods according to the invention, in principle, use can be made of tattoo methods that are known per se. The special feature is that a tattoo ink produced by a method according to the invention, or a tattoo ink according to the invention, is used for the tattooing.

1. A method for producing a tattoo ink, comprising obtaining carbon from at least one of plant, animal, or human material, and adding the carbon to a base composition for tattoo ink.

2. The method as claimed in claim 1, wherein the carbon is obtained from the at least one of the plant, animal, or human material before it is added to the base composition.

3. The method as claimed in claim 2, wherein the material is at least one of the animal or the human material and comprises at least one of hairs, nails, claw material, hoof material, skin, leather, tooth material, or horn.

4. The method as claimed in claim 1, wherein the at least one of the plant, animal, or human material is added to the base composition as powder or as a powder-liquid mixture.

5. The method as claimed in claim 1, wherein the base composition is a liquid or an emulsion or a mixture of at least one liquid or emulsion and at least one solid.

6. The method as claimed in claim 1, wherein the carbon obtained from the at least one of the plant, animal, or human material is present at least in part as graphite or as charcoal.

7. The method as claimed in claim 1, wherein the carbon that is obtained from the at least one of the plant, animal, or human material makes up at least 70% by weight of a mixed material which is added to the base composition.

8. The method as claimed in the carbon obtained from the at least one of the plant, animal, or human material is added to the base composition at a fraction of 0.1% by weight to 50% by weight of the tattoo ink.

9. A tattoo ink, comprising carbon obtained from at least one of plant, animal, or human material that is added to a base composition for tattoo ink.

10. A tattoo method, comprising: using a tattoo ink produced by a method as claimed in claim 1 as the tattoo ink for tattooing, and applying a tattoo.

11. The method as claimed in claim 2, wherein the at least one of the plant, animal, or human material is dead before being added to the base composition.
